SEQUENCE LISTING (3 sequences)

SEQ ID NO: 1 - DNA, 17 bp, Homo Sapiens
ttcgctggagtttcgcc

SEQ ID NO: 2 - DNA, 18 bp, Homo Sapiens
gcttgggggaatagggag

SEQ ID NO: 3 - DNA, 22 bp, Homo Sapiens
aacacgggcattgatctgacgt

(19) United States
(12) Patent Application Publication
(10) Pub. No.: US 2005/0164193 A1
(43) Pub. Date: Jul. 28, 2005

(54) Method for the analysis of methylation patterns within nucleic acids by means of mass spectrometry

(75) Inventor: Kurt Berlin, Stahnsdorf (DE)

Correspondence Address: KRIEGSMAN & KRIEGSMAN, 665 FRANKLIN STREET, FRAMINGHAM, MA, 01702, US

(73) Assignee: Epigenomics AG, Berlin (DE)

(21) Appl. No.: 10/509,145

(22) PCT Filed: Mar. 25, 2003
(86) PCT No.: PCT/EP03/03105

(30) Foreign Application Priority Data
Mar. 25, 2002 (DE) 102 14 232.7
Nov. 7, 2002 (DE) 102 53 068.8

(51) Int. Cl. C12Q 1/68

(52) U.S. Cl. 435/6

(57) ABSTRACT

The present invention describes a method for the analysis of methylation patterns comprising the following steps: a) isolation of genomic nucleic acids from a biological sample, b) amplification of one or more target nucleic acids of said genomic nucleic acids in a manner whereby the methylation patterns of said genomic nucleic acids are maintained in the amplificate nucleic acid, c) performing mass spectrometry on said amplified nucleic acid or fragments thereof to obtain mass spectra; d) evaluating the obtained mass spectra and e) determining the methylation pattern and/or methylation status of the sample. The disclosed invention provides novel methods for the analysis of cytosine methylation patterns within genomic DNA samples. Said method comprises a methylation retaining enzymatic amplification of a test nucleic acid sample, followed by mass spectrometric analysis of the amplificate nucleic acids.

### PRIOR ART

Developments in the field of molecular biology over the last decade have in particular been focused upon the analysis of the human genome. The perceived benefits of the understanding of the functioning of the genome have led to the development of a variety of techniques suitable for the analysis and manipulation of nucleic acid sequences. Of particular interest are techniques that decrease the cost and increase the speed of genetic analysis. One such technique is the application of mass spectrometry to the analysis of nucleic acid sequences.

Mass spectrometry is an analytical technique with multiple applications in the field of chemistry and biology. Its uses include the accurate determination of molecular weights, identification of chemical structures by means of their fragmentation properties, determination of the composition of mixtures and qualitative elemental analysis. In a mass spectrometer a sample is first ionised, different species of ions are then separated according to their mass to charge ratios and the relative abundance of each species of ion is measured.

Of particular utility for the analysis of large molecules are ‘time-of-flight’ (TOF) mass spectrometers which separate ions according to their mass-to-charge ratio by measuring the time it takes the generated ions to travel to a detector. TOF mass spectrometers are advantageous because they are relatively simple, inexpensive instruments with virtually unlimited mass-to-charge ratio range. TOF mass spectrometers have potentially higher sensitivity than scanning instruments because they can record all the ions generated from each ionization event. TOF mass spectrometers are particularly useful for measuring the mass-to-charge ratio of large organic molecules where conventional magnetic field mass spectrometers lack sensitivity. The prior art of TOF mass spectrometers is shown, for example, in U.S. Pat. Nos. 5,045,694 and 5,160,840.

TOF mass spectrometers include an ionization source for generating ions of sample material under investigation. The ionization source contains one or more electrodes or electrostatic lenses for accelerating and focusing the ion beam. In the simplest case the electrodes are grids. A detector is positioned a predetermined distance from the final grid for detecting ions as a function of time. Generally, a drift region exists between the final grid and the detector. The drift region allows the ions to travel, in free flight over a predetermined distance before they impact the detector.

The flight time of an ion accelerated by a given electric potential is proportional to its mass-to-charge ratio. Thus the time-of-flight of an ion is a function of its mass-to-charge ratio, and is approximately proportional to the square root of the mass-to-charge ratio. Assuming the presence of only singly charged ions, the lightest group of ions reaches the detector first and are followed by groups of successively heavier mass groups.

The analysis of biological compounds such as peptides and nucleic acids by mass spectrometry has been hampered by the difficulty in achieving ionisation of large molecules. This has been ameliorated by the development of gentler techniques such as fast atom bombardment (FAB) and electrospray ionization (ESI) collision-induced dissociation/tandem MS.

However the current technique of choice is analysis by means of matrix assisted laser desorption ionisation (MALDI), see for example Karas M, Hillenkamp F. Laser desorption ionization of proteins with molecular masses exceeding 10,000 daltons. Anal Chem. 1988 Oct. 15; 60(20): 2299-301). In this technique the analyte is embedded in a light-absorbing matrix. The matrix is evaporated by a short laser pulse thereby transporting the analyte molecule into the vapour phase in an unfragmented manner. The analyte is ionized by collisions with matrix molecules. An applied voltage accelerates the ions into a field-free flight tube. Due to their different masses, the ions are accelerated at different rates and smaller ions reach the detector sooner than bigger ones. The principle advantages of the technique include a relatively broad mass range, high resolution and sampling rate (up to 1 sample/second). In one aspect MALDI offers a potential advantage over ESI and FAB in that biomolecules of large mass can be ionized and analyzed readily. Furthermore, in contrast to ESI, MALDI produces predominantly singly charged species.

However, although MALDI-TOF spectrometry is well suited to the analysis of peptides and proteins, the analysis of nucleic acids has proved somewhat more difficult (Gut I G, Beck S. DNA and Matrix Assisted Laser Desorption Ionisation Mass Spectrometry. Current Innovations and Future Trends. 1995, 1; 147-57). The problems include a lack of resolution of high molecular weight DNA fragments, DNA instability, and interference from sample preparation reagents.

The sensitivity of the mass spectrometer to nucleic acids is approximately 100 times worse than to peptides and decreases with increasing nucleic acid length. For nucleic acids having a multiply negatively charged backbone, the ionization process via the matrix is considerably less efficient. In MALDI-TOF spectrometry, the selection of the matrix plays an eminently important role. For the desorption of peptides, several very efficient matrixes have been found which produce a very fine crystallisation. However, although several responsive matrixes are suitable for DNA analysis, the difference in sensitivity has not been reduced.

The difference in sensitivity can be reduced by chemically modifying the DNA in such a manner that it becomes more similar to a peptide. Phosphorothioate nucleic acids in which the usual phosphates of the backbone are substituted with thiophosphates can be converted into a chargeneutral DNA using simple alkylation chemistry (Gut I G, Beck S. A procedure for selective DNA alkylation and detection by mass spectrometry. Nucleic Acids Res. 1995 Apr. 25; 23(8): 1367-73). The coupling of a charge tag to this modified DNA results in an increase in sensitivity to the same level as that of peptides. A further advantage of charge tagging is the increased stability of the analysis against impurities which make the detection of unmodified substrates considerably more difficult.

Methods for introducing modified nucleotides that stabilise the nucleic acid against fragmentation have also been described (Schneider and Chait, Nucleic Acids Res, 23, 1570 (1995), Tang et al., J. Am. Soc. Mass Spectrom., 8, 218-224, 1997).

The use of non-cleavable mass tags has also been exploited to address some of the aforementioned deficiencies. For example, Japanese Patent No. 59-131909 discloses a mass spectrometer design that detects nucleic acid fragments separated by electrophoresis, liquid chromatography or high speed gel filtration, wherein atoms have been incorporated into the nucleic acids. The atoms, which normally do not occur in DNA, are sulphur, bromine, iodine, silver, gold, platinum, tin and mercury. See for example, Jacobson K B, Arlinghaus H F, Schmitt H W, Sachleben R A, Brown G M, Thonnard N, Sloop F V, Foote R S, Larimer F W, Woychik R P, et al. ‘An approach to the use of stable isotopes for DNA sequencing.’ Genomics. 1991 January; 9(1): 51-9.

MALDI-TOF analysis has proved to be useful in many aspects of nucleic acid sequence analysis, in particular DNA sequencing. For example Wang et al. (WO 98/03684) have taken advantage of “in source fragmentation” and coupled it with delayed pulsed ion extraction methods for determining the sequence of nucleic acid analytes. Other analysis techniques detect the fragments produced by standard sequencing methods. For example, U.S. Pat. No. 5,288,644 (Beavis, et al.); U.S. Pat. No. 5,547,835 (Koster) and U.S. Pat. No. 5,622,824 (Koster) disclose methods for determining the sequence of a target nucleic acid using MALDI-TOF of ladders of the target produced either by exonuclease digestion or by standard Sanger sequencing methods.

Mass spectrometry has also been used for the analysis of known sequences to determine the presence, location and identity of mutations. U.S. Pat. No. 5,605,798, for example, discloses a method wherein a DNA primer that is complementary to a known target molecule in a region adjacent to the known region of interest is extended with a DNA polymerase in the presence of mass-tagged dideoxynucleotides. The identity of the mutation is then determined by analysing the mass of the dideoxy-extended DNA primer.

One particular advantage of mass spectrometer analysis over other commonly used molecular biological techniques is the ability to analyse multiple samples in a time and cost effective manner and thereby its potential as a high throughput tool. Technological advances such as those outlined in PCT Application WO 95/04160 (Southern, et al.) enable effective analysis of the highly popular oligonucleotide array formats (see for example EP 1138782A2).

The levels of observation that have been studied by the methodological developments of recent years in molecular biology, are the genes themselves, the translation of those genes into RNA, and the resulting proteins. In particular research efforts have focused on the sequences of genes and variations in side sequences. However, the emphasis on fields such as proteomics, genomics and bioinformatics has meant that analysis of epigenetic variations has not received as much scientific attention.

The question of which gene is switched on at which point in the course of the development of an individual, and how the activation and inhibition of specific genes in specific cells and tissues are controlled is correlatable to the degree and character of the methylation of the genes or of the genome. In this respect, pathogenic conditions may manifest themselves in a changed methylation pattern of individual genes or of the genome.

DNA methylation plays a role, for example, in the regulation of the transcription, in genetic imprinting, and in tumorigenesis. The methyl transfer reaction proceeds through a non specific binding of the transferase to the hemimethylated DNA strand, identification of the target base followed by the recruitment of the methyl donor group, most commonly S-adenosyl-L-methionine (AdoMet) to the active site. DNA methyltransferases (m5C Mtase) attach a methyl group to the 5 position carbon. The reaction is carried out via a covalent intermediate between the enzyme and the base whereby the target cytosine is flipped through 180 degrees. The mechanism of methyltransferase dependant cytosine methylation is further reviewed in articles such as Cheng and Roberts ‘AdoMet-dependant methylation, DNA methyltransferases and base flipping’ Nucleic Acids Res. 15; 29(18): 3784-95.

Several species of methyltransferases have been identified, of particular interest to this invention are the family of maintenance methyltransferases that propagate the methylation pattern of hemimethylated DNA within the unmethylated strand, such as Dnmt1. The in vitro action mechanism of DNMT1 is fully discussed in Pradhan, S., Bacolla, A., Wells, R. D., Roberts, R. J. ‘Recombinant Human DNA (Cytosine-5) Methyltransferase. I. Expression, Purification and comparison of de novo and maintenance methylation.’ J. Biol. Chem. 274: 33002-33010 and Bacolla A, Pradhan S, Roberts R. J., Wells R. D. ‘Recombinant human DNA (cytosine-5)methyltransferase. II. Steady-state kinetics reveal allosteric activation by methylated DNA’ J. Biol. Chem. 12; 274(46): 33011-9.

The identification of 5-methylcytosine as a component of genetic information is of considerable interest. However, 5-methylcytosine positions cannot be identified by sequencing since 5-methylcytosine has the same base pairing behaviour as cytosine. Moreover, the epigenetic information carried by 5-methylcytosine is completely lost during PCR amplification. Therefore a new method of analysis has had to have been developed for the analysis of methylation patterns.

Currently the most frequently used method for analyzing DNA for 5-methylcytosine is based upon the specific reaction of bisulfite with cytosine which, upon subsequent alkaline hydrolysis, is converted to uracil which corresponds to thymidine in its base pairing behaviour. An overview of the further known methods of detecting 5-methylcytosine may be gathered from the following review article: Rein, T., DePamphilis, M. L., Zorbas, H., Nucleic Acids Res. 1998, 26, 2255.

The bisulfite treated nucleic acids are then analysed, generally by means of one or more of several methods. For example, amplification of short specific regions of the treated nucleic acid followed by sequencing (Olek A, Walter J. The pre-implantation ontogeny of the H19 methylation imprint. Nat Genet. 1997 November; 17(3): 275-6), assessment of individual cytosine positions by a primer extension reaction (Gonzalgo M L, Jones P A. Rapid quantitation of methylation differences at specific sites using methylation-sensitive single nucleotide primer extension (Ms-SNuPE). Nucleic Acids Res. 1997 Jun. 15; 25(12): 2529-31, WO Patent 9500669) or by enzymatic digestion (Xiong Z, Laird P W. COBRA: a sensitive and quantitative DNA methylation assay. Nucleic Acids Res. 1997 Jun. 15; 25(12): 2532-4). In addition, detection by hybridisation has also been described (Olek et al., WO 99 28498). The use of methylation specific primers for the amplification of bisulphite treated nucleic acids is another commonly used method, and is described in U.S. Pat. No. 6,265,171 to Herman. Other useful methods include the use of oligonucleotide based technologies, such as methylation specific fluorescence-based Real Time Quantitative PCR (described in U.S. Pat. No. 6,331,393) and variations such as the use of dual probe technology (Lightcycler™) or fluorescent amplification primers (Sunrise™ technology). A further suitable method for the use of probe oligonucleotides for the assessment of methylation by analysis of bisulphite treated nucleic acids is the use of blocker oligonucleotides. The use of blocker oligonucleotides has been described in BioTechniques 23(4), 1997, 714-720 D. Yu, M. Mukai, Q. Liu, C. Steinman, although this reference does not describe the application of said tool for the determination of methylation patterns by analysis of bisulphite treated nucleic acids.

However none of the currently used methods for the analysis of methylation patterns directly utilise mass spectrometry, as the analysis of minute samples of nucleic acids is not practical without prior amplification of said sample. No currently known methods of nucleic acid amplification conserve said methylation patterns therefore currently the application of mass spectrometry to the analysis of methylation patterns is not possible on readily available biological samples.

### DESCRIPTION

The method according to the invention provides a means for the analysis of methylation patterns within nucleic acids. Said method enables the assessment of complex methylation patterns by means of a methylation retaining amplification of a nucleic acid sample followed by mass spectrometric analysis. The method according to the invention provides improvements over the state of the art in that it is particularly suited to the medium or high throughput analysis of biological samples.

It is an object of the present invention to provide a method for the analysis of methylation patterns comprising the following steps:
a) isolation of genomic nucleic acids from a biological sample,
b) amplification of one or more target nucleic acids of said genomic nucleic acids in a manner whereby the methylation patterns of said genomic nucleic acids are maintained in the amplificate nucleic acid,
c) performing mass spectrometry on said amplified nucleic acid or fragments thereof to obtain mass spectra;
d) evaluating the obtained mass spectra and
e) determining the methylation pattern and/or methylation status of the sample.

According to the invention it is preferred that in step a) the genomic DNA is obtained from cells or cellular components which contain DNA, sources of DNA comprising, for example, cell lines, biopsies, blood, sputum, stool, urine, cerebral-spinal fluid, tissue embedded in paraffin such as tissue from eyes, intestine, kidney, brain, heart, prostate, lung, breast or liver, histologic object slides, and all possible combinations thereof.

According to the invention is also preferred that step b) is carried out by means of the following additional steps or sub-steps:
i) amplification of the target genomic nucleic acid sequence in a semiconservative manner,
ii) methylation of the synthesised strand whereby the 5′ cytosine methylation status of the CG dinucleotides in the template strand is copied to the CG dinucleotides of the synthesised strand.

In this case it is especially preferred that the method is further comprising the following steps:
iii) denaturation of the double stranded nucleic acids to form single stranded nucleic acids,
iv) repetition of steps i) to iii) until a desired number of amplificates is obtained.

Therein it is also preferred that in step i) the method of amplification is selected from: ligase chain reaction, polymerase chain reaction, polymerase reaction, rolling circle replication.

It is especially preferred that in step ii) said methylation is carried out by enzymatic means. A preferred enzyme is a maintenance methyltransferase.

According to the present invention it is also preferred that the methyltransferase is DNA (cytosine-5) Methyltransferase (DNMT 1).

Preferred is also according to the invention that the methyl group is obtained from the donor molecule S-adenosylmethionine.

In a preferred embodiment of the present invention step b) of the method is carried out by means of the following additional steps or sub-steps
(1) heating the genomic DNA to a temperature operative to cause denaturation,
(2) cooling the denatured DNA in the presence of single stranded oligonucleotide primers such that the primers anneal to the DNA,
(3) heating the mixture in the presence of a polymerase and nucleotides to a temperature such that the primers are extended,
(4) contacting the double stranded nucleic acid with enzymes and/or agents under conditions conducive to the methylation of the synthesised strand such that the CpG dinucleotides within the synthesised strand are methylated according to the methylation status of the corresponding CpG dinucleotide on the template strand thereby preserving the genomic methylation pattern,
(5) repeating steps (1) to (4) a desired number of times to reach a desired number of nucleic acids.

In the method according to the present invention it is preferred that the amplificate nucleic acids are fragmented prior to step C. Therein it is further preferred that said fragmentation is carried out by enzymatic or chemical means.

In the method according to the present invention it is preferred that step c) is carried out by means of time-of-flight MALDI or ESI mass spectrometry.

It is also preferred that in step c) internal and/or external calibration is used.

It is also preferred according to the invention that prior to step c) the nucleic acids are purified. Therein it is especially preferred that wherein said nucleic acids are single stranded.

It is also preferred according to the invention that the amplificate nucleic acids are less than 100 base pairs in length.

According to the invention it is also preferred that any primer oligonucleotides used during step b) do not contain CG dinucleotides.

It is also especially preferred according to the invention that said amplificates are immobilised upon a solid phase.

A method according to the invention is also preferred, wherein the synthesised amplificates comprise at least one chemical modification of an internucleoside linkage, a sugar backbone, or a nucleoside base.

A method according to the present invention is also preferred wherein steps d) and e) are carried out as follows:
d) comparing the obtained mass spectra with reference mass spectra obtained of the nucleic acid in its fully methylated and/or fully unmethylated form and
e) determining by said comparison whether fragments are methylated, unmethylated or partially methylated and thereby determining the methylation pattern of the nucleic acid.

In another preferred embodiment of the present invention it is likewise preferred that steps d) and e) are carried out as follows:
d) determining the molecular weight of the fragment or fragments
e) determining the methylation status of said fragments.

Preferred is also according to the invention that the methyl group carries a detectable label which is incorporated into the synthesised nucleic acid strand.

Preferred is also according to the invention that a mass label is incorporated into the amplificate nucleic acids.

It is another object of the present invention to use of a method according to the invention as described above for the analysis of methylation patterns within genomic DNA.

A third object of the present invention is a kit for analysis of methylation within nucleic acids according to a method of the present invention comprising
reagents for the methylation retaining amplification of genomic DNA,
reagents for the mass spectrometric analysis of nucleic acids.

In other words, the objective of the invention is achieved by means of a method comprising the following steps:
a) isolation of genomic nucleic acids from a biological sample,
b) amplification of one or more target nucleic acids of said genomic nucleic acids in a manner whereby the methylation patterns of said genomic nucleic acids are maintained in the amplificate nucleic acid,
c) performing mass spectrometry on said amplified nucleic acid or fragments thereof to obtain a mass spectrum.

In a particularly preferred embodiment the results of the mass spectrometric analysis are then analysed either by comparison to a signature spectrum and/or by analysis of the molecular weight of the produced fragments. From the analysis or analyses the methylation pattern of the genomic region of interest is deduced.

In the first step of the method genomic DNA is isolated from a biological sample. Suitable sources include, but are not limited to, cells or cell components, for example, cell lines, biopsies, blood, sputum, stool, urine, cerebro-spinal fluid, tissue embedded in paraffin such as tissue from eyes, intestine, kidney, brain, heart, prostate, lung, breast or liver, histological slides, or combinations thereof.

The DNA is then extracted by means that are standard to one skilled in the art, these include the use of detergent lysates, sonification and vortexing with glass beads. Once the nucleic acids have been extracted the genomic double stranded DNA is used in the analysis.

In the second step of the method one or more preselected regions of the genomic DNA are amplified in a manner whereby the methylation patterns of said genomic DNA are maintained in the amplificate nucleic acids.

In the third step of the method, the amplificate or fragments thereof are analysed by mass spectrometry. The methylation pattern of the genomic nucleic acid is then determined by analysis of the mass spectrum. Preferably this is by time of flight mass spectrometry using the MALDI or ESI methods.

In a preferred embodiment the second step of said method comprises the following steps.

In the first step of the method semiconservative replication of the target genomic nucleic acid sequence is carried out such that the resultant amplificate is a hemimethylated nucleic acid. Any method of in vitro semiconservative replication may be utilised.

Said methods for the amplification of specific DNA targets are based upon template directed oligonucleotide primer extension by polymerases. Particularly preferred are the enzymatic methods of isothermal replication (also known as rolling circle replication), ligase based reactions and polymerase based reactions.

The most widely utilised of these methods is the polymerase chain reaction (Mullis, K. et al., Cold Spring Harbor Symp. Quant. Biol. 51: 263-273 (1986); Erlich H. et al., EP 50,424; EP 84,796, EP 258,017, EP 237,362; Mullis, K., EP 201,184; Mullis K. et al., U.S. Pat. No. 4,683,202; Erlich, H., U.S. Pat. No. 4,582,788; Saiki, R. et al., U.S. Pat. No. 4,683,194 and Higuchi, R. “PCR Technology,” Ehrlich, H. (ed.), Stockton Press, NY, 1989, pp 61-68). In the first step the DNA double helix is denatured by transient heating. This is followed by the annealing of two species of primers, one to each strand of DNA. Subsequently the annealed primers are extended using a polymerase dNTPs.

Other suitable methods include the ligase chain reaction and variants thereof. In the ligase chain reaction two probe oligonucleotides are hybridised to a single stranded template nucleic acid such that the 5′ end of one oligonucleotide probe hybridises next to the 3′ end of the other oligonucleotide probe thereby allowing the two oligonucleotides to be joined together by a ligase. In a variant of the ligase reaction wherein said ends do not lie adjacent to one another, the gap between the two oligonucleotides may be ‘filled in’ by the action of a polymerase.

Both the ligase and polymerase reactions are generally performed as chain reactions by performing a denaturation step. The two strands are heat denatured thereby allowing the resultant single stranded nucleic acid to be used as a template nucleic acid in subsequent cycles of ligase or polymerase enabled nucleic acid replication allowing the reaction cycles to be repeated the desired number of times.

According to the method of the invention any of said ‘chain reaction methods’ may be utilised by carrying out a methylation step that will be described below, prior to the denaturation step of the method.

Rolling circle isothermal nucleic acid amplification is enabled by means of the circularisation of the target nucleic acid, hereinafter referred to as an amplification target circle (ATC). The circularised nucleic acid is then isothermally replicated using rolling circle replication primers and a polymerase enzyme. There are no denaturation and annealing stages, hence DNA replication is both continuous and isothermal. The resultant DNA comprises a catenated linear DNA of identical sequence to the ATC. Several variants of the method have been described, see for example U.S. Pat. No. 5,871,921 (Landegren et al.).

The design of primers for the replication and or amplification of the genomic DNA using the above described methods should be obvious to one skilled in the art. These should include at least two oligonucleotides whose sequences are each reverse complementary or identical to an at least 18 base-pair long segment flanking the base sequences to be analysed. In a particularly preferred embodiment said primers are designed so as to amplify a nucleic acid sequence of not more than 100 base pairs in length. Furthermore, said primer oligonucleotides are preferably characterised in that they do not contain any CpG dinucleotides.

In the next step of the method the newly synthesised unmethylated strand of the double stranded nucleic acid is selectively methylated. Said methylation reaction is carried out on specific cytosine bases of CG dinucleotides by reference to the methylation state of the template strand of the nucleic acid. Wherein a CG dinucleotide is methylated on the template strand, the cytosine on the newly synthesised strand which is hybridised to the guanine of said dinucleotide is methylated at the 5′ position.

In a preferred embodiment of the method this is carried out by contacting the double stranded nucleic acid with a methyltransferase enzyme and a methyl donor molecule under conditions conducive to the methylation of the synthesised strand. The methylation action of said enzymes being such that the CpG dinucleotides within the synthesised strand are methylated according to the methylation status of the corresponding CpG dinucleotide on the template strand thereby preserving the genomic methylation pattern.

In a preferred embodiment of the present invention the methyltransferase is a maintenance methyltransferase. Suitable methylation enzymes for use in Step D of the method are limited to those capable of methylating the cytosine at the 5 position according to the methylation status of the cytosine within the corresponding CpG dinucleotide on the template strand. In the case of a cytosine within a CpG upon the template strand being methylated, then the corresponding CpG to which it is hybridised on the synthesised strand will be methylated by action of the enzyme at the 5′ position of the cytosine base. If the cytosine within said CpG is unmethylated then the corresponding CpG on the synthesised strand will remain unmethylated. The reaction is carried out using appropriate buffers and other reagents and reaction conditions as recommended by the supplier of the enzyme, this may include a methyl donor molecule such as, but not limited to S-adenosylmethionine. The enzyme may be from any source e.g. Human, mouse, recombinant. In a particularly preferred embodiment the methyltransferase is DNA (cytosine-5) Methyltransferase (DNMT 1).

According to the invention it is preferred that the methyl donor molecule is S-adenosylmethionine.

It is also preferred according to the invention that the methyl group carries a detectable label which is incorporated into the synthesised nucleic acid strand.

In a further preferred embodiment of the method subsequent to the nucleic acid replication and methylation steps a denaturation step is carried out. Preferably this is in the form of a heat denaturation wherein the double stranded nucleic acid is heated to a temperature operative to break the hybridisation bonds between the two strands. Suitable temperatures should be obvious to one skilled in the art and are dependant upon the length of the amplificate and the GC content of the nucleic acids.

Said denatured strands are then used as template nucleic acids for a further round of template directed oligonucleotide extension followed by the above described methylation steps and denaturation steps. Said steps may be carried out a desired number of times in order to amplify the number of copies of the target nucleic acid to a desired level, a manner akin to the polymerase chain reaction or the ligase chain reaction.

In a particularly preferred embodiment said method comprises the following step.
(a) heating the genomic DNA to a temperature operative to cause denaturation
(b) cooling the denatured DNA in the presence of single stranded oligonucleotide primers such that the primers anneal to the DNA
(c) heating the mixture in the presence of a polymerase and nucleotides to a temperature such that the primers are extended
(d) contacting the double stranded nucleic acid with a methyltransferase and a methyl donor molecule under conditions conducive to the methylation of the synthesised strand such that the CpG dinucleotides within the synthesised strand are methylated according to the methylation status of the corresponding CpG dinucleotide on the template strand thereby preserving the genomic methylation pattern
(e) repeating steps A-D a desired number of times to reach a desired number of nucleic acids.

Reaction temperatures suitable for each stage of the process will be obvious to one skilled in the art, as they are analogous to those used in standard polymerase chain reactions. Typically these will be in the region of 94° C. for denaturation, 55° C. for annealing, and 72° C. for extension. It is also possible to combine the annealing and extension incubations to yield a two temperature incubation process, typically around 94° C. for denaturation, and around 50°-65° C. for an annealing and extension incubation. The optimal incubation temperatures and times differ, however, with different targets and primer oligonucleotides as the operative temperatures are dependant on factors such as nucleic acid length and G/C content.

In the third step of the method the amplificates are analysed by means of mass spectroscopy. More preferably this is by ESI (electron spray ionization) or MALDI-TOF (matrix assisted laser desorption ionization time of flight) mass spectrometry.

Prior to the mass spectrometric analysis of the amplificates, several modifications may be made to the amplificate nucleic acid to facilitate detectability in the mass spectrometer. Said modifications include but not are not limited to, modifications to the internucleoside linkages, sugar backbone or bases. Said modifications may be carried out both during or post synthesis of the amplificate.

It is a particularly preferred embodiment of the method that prior to the mass spectrometric analysis of the amplificate nucleic acid said nucleic acids are fragmented in order to obtain better detectability in the mass spectrometer. More preferably said fragmentation is carried out in a sequence specific manner. Methods of fragmentation of nucleic acids will be known to one skilled in the art.

Sequence specific fragmentation of the amplificates may be achieved by either chemical or enzymatic means. Enzymatic cleavage of nucleic acids may be achieved by a variety of ribo and deoxyribonucleases or glycosylases. The advantage being that prior knowledge of the sequence of the fragment would allow the fragmentation of the amplificate into predefined fragments, preferably with ‘blunt’ ends (wherein no 3′ or 5′ single stranded overhangs are present post cleavage).

Chemical cleavage is less costly and a more robust assay, but is less site specific. Chemically modified internucleoside bonds inserted at specified locations within the amplificate allow for a predefined fragmentation and MALDI-TOF detection. Examples of cleavable nucleotide modification include the ‘achiral’ 5′ phosphoramidate analogue, available in a triphosphate form, capable of utilisation by some polymerases and wherein cleavage occurs under the acidic conditions of many commonly used MALDI matrices.

Another useful modification that is a preferred embodiment of the invention is the incorporation of deoxyuridine into the amplificate nucleic acid, digestion with uracil-N-glycosidase thereby results in the fragmentation of the nucleic acid.

Another preferred embodiment useful for facilitating detectability in the mass spectrometer base is the incorporation of 7-desa-guanosine and 7-desa-adenosine into the amplificate. These compounds are reported to stabilise the nucleic acid during mass spectrometry.

In a further preferred embodiment of the method the sugar backbone of the amplificate nucleic acid may be modified according to the teachings of Gut et al. U.S. Pat. No. 6,268,129. In this method the amplificate nucleic acid is synthesised using phosphorothioate linkage or a phosphoroselenoate linkage between the sugar residues and and a positively charged tag or moiety. The synthesised amplificate is then alkylated to eliminate the charge on the phosphorothioate linkage or phosphoroselenoate linkage thereby enabling the person skilled in the art to selectively charge the backbone of the nucleic acid.

The sensitivity of the technique may also be improved by the use of internal and/external calibrants, as known in the art. Calibrants are analytes of known mass that are analyzed by the mass spectrometer and are used as reference analytes to improve the mass accuracy and precision of the analysis of an unknown compound. Internal calibrants are included within the sample to be analysed and are thereby simultaneously analysed whereas external calibrants are not included in the sample and are analysed separately.

Prior to the mass spectrometric analysis of the amplificate it may be desirable to purify the amplificate, to remove contaminants such as polymerase, salts, primers and triphosphates. This may be by any means that are standard in the art including precipitation, ion exchange spin columns, filtering, dialysis and ion exchange chromatography. A particularly preferred method is the use of (magnetic) glass beads to precipitate nucleic acids of a specific size range and allow them to be rigorously washed. One such commonly used technique which is suitable is the labelling of the amplification primers with biotin. The biotin labelled strand is then captured by binding it to streptavidin, the single stranded nucleic acids are then used in the analysis.

In a particularly preferred embodiment the methylation pattern of the genomic nucleic acid is determined by analysis of the obtained mass spectrum.

In a particularly preferred embodiment of the method the mass spectrum obtained is compared to the mass spectrum of fragments obtained from known samples of either methylated or unmethylated versions of the target nucleic acid. These known spectra are referred to as “reference” spectra. A simple comparison of the sample spectrum vs. reference spectra enables the determination of the methylation status of the sample.

In a further embodiment of the method the mass to charge ratio of the amplificate or fragments thereof are used to determine the molecular weight of each of said nucleic acids and thereby determine its methylation state. However this method is only enabled wherein the sequence of said nucleic acids is known.

In a further embodiment of the method the nucleic acids may be immobilised upon a solid surface, at high density prior to mass spectrometric analysis. In one embodiment, the primer oligonucleotide is immobilised, directly or by means of a cross-linking agent, to a solid support. The amplification and methylation reactions are then carried out upon the solid surface.

Preferred solid supports are those which can support linkage of nucleic acids thereto at high densities. A variety of insoluble support materials may be utilised including, but not limited to silica gel, cellulose, glass fibre filters, glass surfaces, metal surfaces (steel, gold, silver, aluminium, silicon and copper), plastic materials (e.g., of polyethylene, polypropylene, polyamide, polyvinyldenedifluoride) and silicon.

Any linker known to those of skill in the art suitable for immobilising nucleic acids to solid supports for mass spectrometric analysis may be used. Preferred linkers are selectively cleavable linkers, acid cleavable linkers, such as bismaleimidoethoxy propane, acid labile, photocleavable and heat sensitive linkers.

In a particularly preferred embodiment, the nucleic acid is immobilised using a photocleavable linker moiety that is cleaved during mass spectrometry. Exemplary photolabile cross-linkers include, but are not limited to, 3-amino-(2-nitrophenyl)propionic acid (Rothschild et a. (1996) Nucleic Acids Res. 24: 361-66).

In an alternative preferred embodiment the linker moiety may be a chemically cleavable linker molecule. In this case acid-labile linkers are particularly preferred for mass spectrometry, especially MALDI-TOF MS, because the acid labile bond is cleaved during conditioning of the nucleic acid upon addition of the 3-HPA (3-hydroxy picolinic acid) matrix solution, for example.

In embodiments of the methods in which a cross-linking reagent is not employed, a modified primer oligonucleotide may be attached to a solid surface by reaction directly with an appropriately functionalized surface to yield an immobilised nucleic acid. Thus, for example, an iodoacetyl-modified surface (or other thiol-reactive surface functionality) can react with a thiol-modified nucleic acid to provide immobilised nucleic acids.

Multiple species of primer oligonucleotides may be arranged on a plane solid phase in the form of a rectangular or hexagonal lattice, also known as an ‘array’.

An overview of the Prior Art in oligomer array manufacturing can be gathered from a special edition of Nature Genetics (Nature Genetics Supplement, Volume 21, January 1999), published in January 1999, and from the literature cited therein.

Furthermore, a subject matter of the present invention is a kit which is preferably composed of two sets of reagents. A first set of reagents required for the methylation retaining amplification of target nucleic acids, accordingly this may include polymerase and/or methyltransferase enzymes, primer oligonucleotides and methyl donor reagents. A second set of reagents provides reagents required for the mass spectrometric analysis of the nucleic acid amplificates, for example but not limited to a suitable matrix material for MALDI analysis.

As used herein, the term “nucleic acid” refers to oligonucleotides or polynucleotides such as deoxyribonucleic acid (DNA) and ribonucleic acid (RNA) as well as analogs of either RNA or DNA, for example, made from nucleotide analogs, any of which are in single or double-stranded form or may form triple helices. Nucleic acid molecules can be synthetic or can be isolated from a particular biological sample using any number of procedures which are well-known in the art, the particular procedure chosen being appropriate for the particular biological sample.

As used herein, nucleotides include nucleoside mono-, di-, and triphosphates. Nucleotides also include modified nucleotides such as phosphorothioate nucleotides and deazapurine nucleotides.

The phrase ‘methylation pattern’ as used herein is taken to mean the specific consecution of 5′ methyl groups attached to cytosine bases within a nucleic acid, more specifically, wherein said cytosine bases are present as part of a CG dinucleotide.

The phrase ‘methylation retaining amplification’ as used herein is taken to mean the amplification of a nucleic acid wherein the methylation pattern of the template nucleic acid is conserved in copies of said template nucleic acid.

‘Mass spectrum’ refers to a graphical representation of ionic species separated according to their mass-to-charge ratios. The spectrum is a plot of m/z versus measured ion abundance.

The phrase ‘target nucleic acid’ refers to a nucleic acid sequence to be amplified and analysed according to the methods disclosed herein.

The phrase ‘template directed oligonucleotide primer extension’ refers to the replication of a single stranded sequence by means of hybridisation of a primer oligonucleotide, preferably to the 3′ end of said sequence, followed by the enzymatic extension of said primer by enzymatic means.

‘Semiconservative replication’ as used herein means the replication of a template nucleic acid wherein each copy of said nucleic acid is comprised of the template strand and a synthesised strand.

The phrase ‘template strand’ refers to the single stranded nucleic acid which serves as a template for a nucleic acid amplification or replication by means of a template directed oligonucleotide primer extension reaction. Said phrase also refers to the use of said nucleic acid during successive cycles of said enzymatic process e.g. polymerase chain reaction, ligase chain reaction.

The phrase ‘synthesised strand’ refers to the product of a template directed oligonucleotide primer extension reaction wherein said nucleic acid has not in itself served as a template in a template directed oligonucleotide primer extension reaction.

“Amplification” of nucleic acids or polynucleotides is any method that results in the formation of one or more copies of a nucleic acid or polynucleotide molecule (exponential amplification) or in the formation of one or more copies of only the complement of a nucleic acid or polynucleotide molecule (linear amplification).

### EXAMPLES

### Example 1

Methylation Retaining PCR Amplification

Genomic DNA commercially available from Promega is used in the analysis. A CpG rich fragment of the regulatory region of the GSTPi gene is used in the analysis. The DNA is firstly artificially methylated at all cytosine 5 positions within the CpGs (upmethylation). The upmethylated DNA is then amplified using one round of PCR. The resultant amplificate is then divided into two samples, Sample A (the control sample) is amplified using conventional PCR. Sample B is amplified according to the disclosed method. The two samples are then compared in order to ascertain the presence of methylated CpG positions within Sample B. The comparison is carried out by means of a bisulphite treatment and analysis of the treated nucleic acids.

Upmethylation

Reagents:

DNA
SssI Methylase (concentration 2 units/μl).
SAM (S-adenosylmethionine)
4.5 μl Mss1-Buffer (NEB Buffer B+ (10 mMole Tris-HCl 300 mMole NaCl, 10 mMole Tris-HCl, 0.1 mMole EDTA, 1 mMole dithiothreitol, 500 μg/ml BSA, 50% glycerol (pH 7.4 at 25° C.) pH 7.5; 10 mMole MgCl2; 0.1 mg/ml BSA)
dd water (0.2 μm-filtered autoclaved, DNases, RNases, proteases, phosphatases free).
   Method:

Reagents are combined and incubated at 37° C. for 16 hours. The sample may then be stored in the refrigerator (+4° C.).

The upmethylated DNA is digested using the restriction enzyme.

PCR

Reagents:

primer I: TTCGCTGGAGTTTCGCC (SEQ ID NO:1)
primer II: GCTTGGGGGAATAGGGAG (SEQ ID NO:2)
HotStart Taq Polymerase (QIAGEN)
10× PCR buffer (QIAGEN)
dNTP solution (25 mMole each)
water (0.2 μm-filtered, autoclaved, DNases, RNases, proteases, phosphatases free).

Reagents are to be combined in a reaction solution in the order above. The reaction solution is then cycled in a thermalcycler according to the following. An initial denaturation at 95° C. is carried out for 15 min. This is followed by primer annealing at 55° C. for 45 sec. and elongation at 72° C. for 1.5 min.

The resultant reaction solution is then divided into two equal samples, A and B. Each sample is treated as below.

Sample A

Standard PCR as described above. The reaction is cycled for 40 cycles at 95° C. for 1 minute, 55° C. for 45 sec. and elongation at 72° C. for 1.5 min.

Sample B

Reagents:

Human DNA (cytosine-5) Methyltransferase (New England Biolabs)
Dnmt 1 reaction buffer (50 mMole Tris HCL pH7.8, 1 mMole EDTA, 1 mMole dithiothreitol, 7 μg/ml PMSF, 5% glycerol) 100 μg/ml BSA

Steps 1 to 4 are repeated 40 times:

1. DNA is precipitated and pelleted, resuspended using Dnmt1 reaction buffer, DNMT and BSA.

2. The reaction solution is incubated at 37° C.

3. DNA is precipitated and pelleted, resuspended using PCR reagents as above.

4. One cycle of PCR is carried out at 95° C. for 1 minutes, 55° C. for 45 sec. and elongation at 65° C. for 2 min.

Sample Analysis

Both samples are analysed in order to ascertain their relative levels of methylation. In a first step the two samples are treated in order to distinguish between methylated and non methylated cytosines. The treatment is carried out using a solution of sodium-disulfite. The treatment converts cytosine to thymine while preserving 5-methyl-cytosine as cytosine. Sample A is thereby thymine rich relative to Sample B, which is relatively cytosine rich. Following bisulphite treatment both samples are analysed by means of sequencing in order to ascertain their degree of methylation (i.e. relative concentrations of cytosine and thymine). Sequencing is carried out by means of the Sanger method using the ABI 310 sequencer (Applied Biosystems).

### Example 2

Mass Spectrometric Analysis of a Methylated Oligonucleotide

A test sample (not from a patient) of an oligonucleotide of known sequence and unknown methylation status is provided. It is required that the methylation status of the sample be ascertained, this may be fully methylated, fully unmethylated or a mixture of the two. The sequence of the oligonucleotide is AACACGGGCATTGATCTGACGT (SEQ ID NO: 3).

Reference Spectrum

In order to correctly identify the methylation status of the sample it was necessary to ascertain the spectra of two control samples. Accordingly an oligonucleotide according to SEQ ID NO:3 was ordered from a commercial supplier, one sample being methylated at each cytosine within the oligonucleotide and the other being fully unmethylated. A sample of the unmethylated oligonucleotide was analysed by MALDI-TOF mass spectrometry and the resultant spectrum can be seen in FIG. 1, the mass of the oligonucleotide was measured as 6757.97 daltons. The fully methylated sample was them analysed by means of MALDI-TOF mass spectrometry and the resultant spectrum can be seen in FIG. 2, the mass of the oligonucleotide was measured as 6785.65 daltons.

Mass Spectrometric Analysis of Provided Sample

The test sample was analysed by means of MALDI-TOF mass spectrometry and the resultant spectrum can be seen in FIG. 3. Two peaks were observed at 6758.01 daltons and 6788.70 daltons. As can be seen by comparison to FIGS. 1 and 2, the sample matter contained two species of oligonucleotides, a first corresponding to the fully methylated sample, and a second corresponding to the fully unmethylated sample. It was thereby deduced that the sample was a mixture of the two species of oligonucleotides, this was confirmed by the supplier of the test sample. All mass spectrometric analyses were carried out using a standard matrix of 0.1 Mole diammonium citrate and 3′-Hydroxypicolinic acid in acetonitril combined in a 1:1 ratio. The analysis was carried out by a Bruker Biflex™ III mass spectrometer. Analysis of the spectra was carried out using the Bruker software XACQ 4.0.4, subsequent editing was carried out using the Bruker X-TOF 5.1.0 software. No additional purification was required, as all oligonucleotides were provided in a salt solution by suppliers. The adducts formed between the K and Na salts in the solution and the oligonucleotides accounted for the additional peaks that can be seen in the spectrum. For the analysis, 0.5 μl of 100 pMol/L oligonucleotide solution was combined with 0.5 μl of matrix and spotted upon the target.

### FIGURES

FIG. 1 shows the mass spectrum of the oligonucleotide analysed according to Example 1. The X-axis shows the abundance of each species of ion, the Y-axis shows the mass of each species. Additional peaks are observed wherein adducts are formed between salts present in the solution and the oligonucleotides. The analysed oligonucleotide contains no methylated cytosines and the observed mass is 6757.97 daltons.

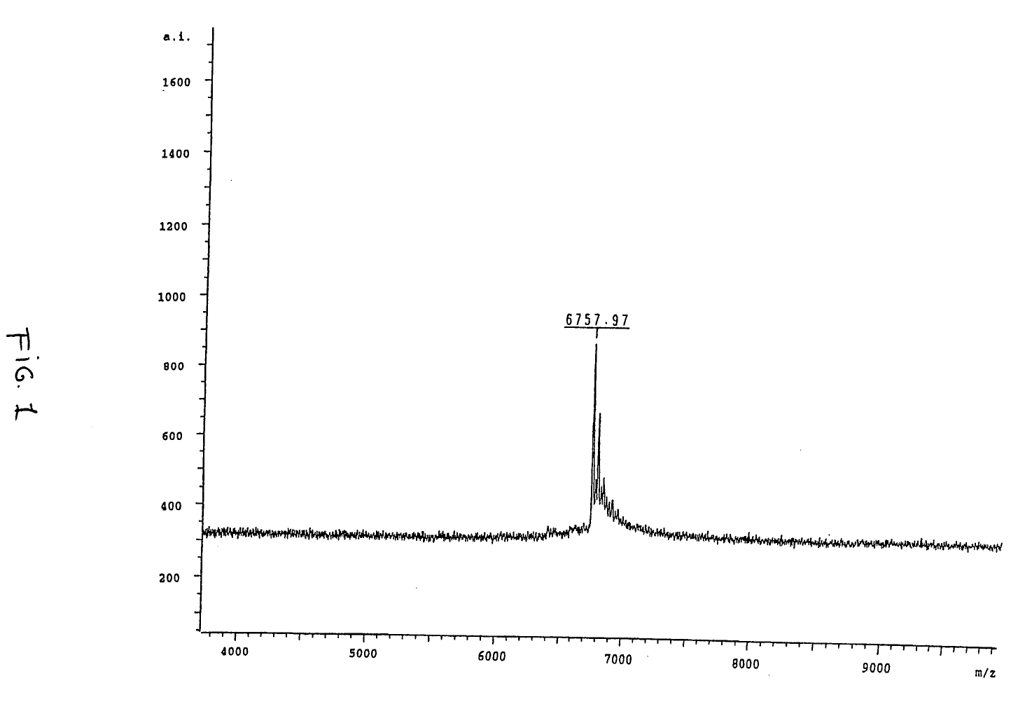

FIG. 2 shows the mass spectrum of the oligonucleotide analysed according to Example 1. The X-axis shows the abundance of each species of ion, the Y-axis shows the mass of each species. Additional peaks are observed wherein adducts are formed between salts present in the solution and the oligonucleotides. The analysed oligonucleotide contains is fully methylated at all cytosines and the observed mass is 6785.65 daltons.

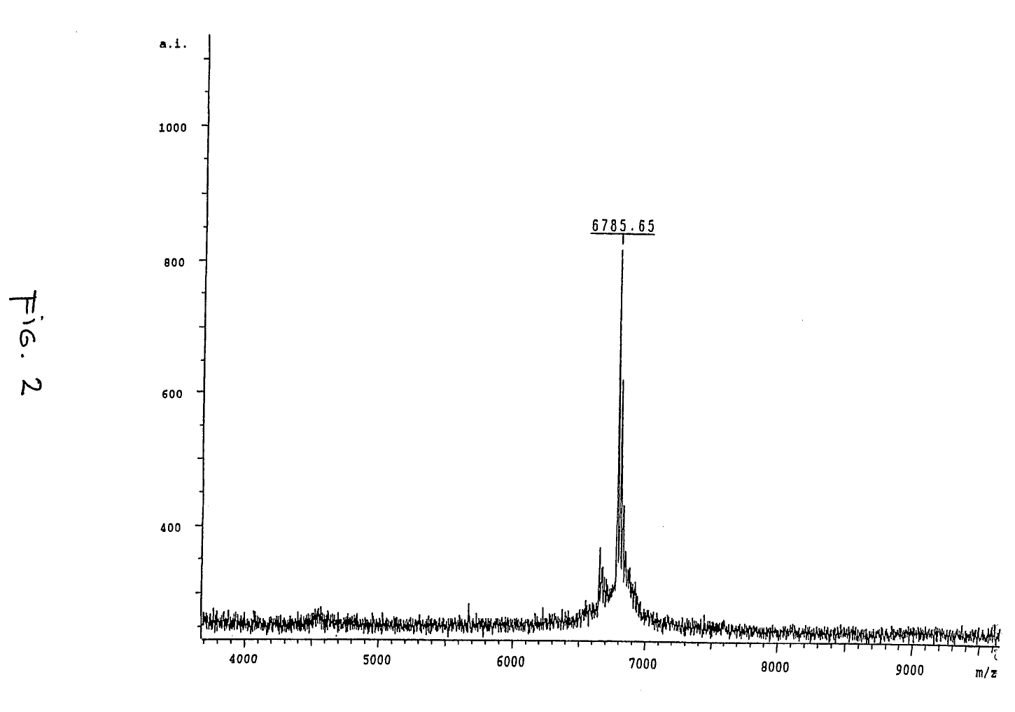

FIG. 3 shows the mass spectrum of the test sample analysed according to Example 3. The X-axis shows the abundance of each species of ion, the Y-axis shows the mass of each species. Additional peaks are observed wherein adducts are formed between salts present in the solution and the oligonucleotides. Peaks are observed at 6758.01 and 6788.70 daltons. Thereby it was concluded that the sample contained a mixture of both methylated and unmethylated oligonucleotides.

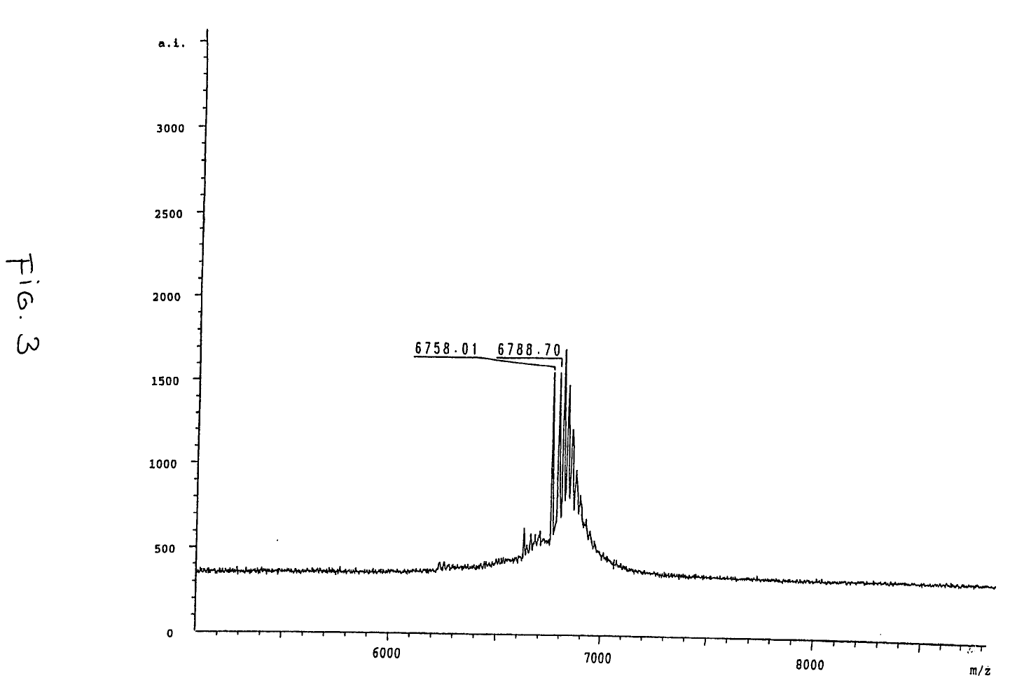

1. A method for the analysis of methylation patterns comprising the following steps:
(a) isolation of genomic nucleic acids from a biological sample,
(b) amplification of one or more target nucleic acids of said genomic nucleic acids in a manner whereby the methylation patterns of said genomic nucleic acids are maintained in the amplificate nucleic acid,
(c) performing mass spectrometry on said amplified nucleic acid or fragments thereof to obtain mass spectra;
(d) evaluating the obtained mass spectra, and
(e) determining the methylation pattern and/or methylation status of the sample.

2. A method according to claim 1, characterised in that in step a) the genomic DNA is obtained from cells or cellular components which contain DNA, sources of DNA comprising, for example, cell lines, biopsies, blood, sputum, stool, urine, cerebral-spinal fluid, tissue embedded in paraffin such as tissue from eyes, intestine, kidney, brain, heart, prostate, lung, breast or liver, histological object slides, and all possible combinations thereof.

3. A method according to claim 1, characterised in that step b) is carried out by means of the following additional steps or sub-steps:
(i) amplification of the target genomic nucleic acid sequence in a semiconservative manner,
(ii) methylation of the synthesized strand whereby the 5′ cytosine methylation status of the CG dinucleotides in the template strand is copied to the CG dinucleotides of the synthesized strand.

4. A method according to claim 3, characterised in that it is further comprising the following steps:
iii) denaturation of the double stranded nucleic acids to form single stranded nucleic acids,
iv) repetition of steps i) to iii) until a desired number of amplificates is obtained.

5. A method according to claim 3, wherein in step i) the method of amplification is selected from: ligase chain reaction, polymerase chain reaction, polymerase reaction, rolling circle replication.

6. A method according to claim 3, wherein in step ii) said methylation is carried out by enzymatic means.

7. A method according to claim 6 wherein said enzyme is a maintenance methyltransferase.

8. A method according to claim 6, wherein the methyltransferase is DNA (cytosine-5) Methyltransferase (DNMT 1).

9. A method according to claim 6, wherein the methyl group is obtained from the donor molecule S-adenosylmethionine.

10. A method according to claim 1, wherein step b) is carried out by means of the following additional steps or sub-steps
(1) heating the genomic DNA to a temperature operative to cause denaturation,
(2) cooling the denatured DNA in the presence of single stranded oligonucleotide primers such that the primers anneal to the DNA,
(3) heating the mixture in the presence of a polymerase and nucleotides to a temperature such that the primers are extended,
(4) contacting the double stranded nucleic acid with enzymes and/or agents under conditions conducive to the methylation of the synthesised strand such that the CpG dinucleotides within the synthesised strand are methylated according to the methylation status of the corresponding CpG dinucleotide on the template strand thereby preserving the genomic methylation pattern,
(5) repeating steps (1) to (4) a desired number of times to reach a desired number of nucleic acids.

11. A method according to claim 1, wherein the amplificate nucleic acids are fragmented prior to step C.

12. A method according to claim 11, wherein said fragmentation is carried out by enzymatic or chemical means.

13. A method according to claim 1, wherein step c) is carried out by means of time-of-flight MALDI or ESI mass spectrometry.

14. A method according to claim 1, wherein in step c) internal and/or external calibration is used.

15. A method according to claim 1, wherein, prior to step c) the nucleic acids are purified.

16. A method according to claim 15, wherein said nucleic acids are single stranded.

17. A method according to claim 1, wherein the amplificate nucleic acids are less than 100 base pairs in length.

18. A method according to claim 1, wherein any primer oligonucleotides used during step b) do not contain CG dinucleotides.

19. A method according to claim 1, wherein said amplificates are immobilised upon a solid phase.

20. A method according to claim 1, wherein the synthesised amplificates comprise at least one chemical modification of an internucleoside linkage, a sugar backbone, or a nucleoside base.

21. A method according to claim 1, characterised in that steps d) and e) are carried out as follows:
d) comparing the obtained mass spectra with reference mass spectra obtained of the nucleic acid in its fully methylated and/or fully unmethylated form and
e) determining by said comparison whether fragments are methylated, unmethylated or partially methylated and thereby determining the methylation pattern of the nucleic acid.

22. A method according to claim 1 characterised in that steps d) and e) are carried out as follows:
d) determining the molecular weight of the fragment or fragments
e) determining the methylation status of said fragments.

23. A method according to claim 1, wherein the methyl group carries a detectable label which is incorporated into the synthesised nucleic acid strand.

24. A method according to claim 1, wherein a mass label is incorporated into the amplificate nucleic acids.

25. The use of a method according to claim 1 for the analysis of methylation patterns within genomic DNA.

26. A kit for analysis of methylation within nucleic acids according to any one of claims 1 to 25 comprising
reagents for the methylation retaining amplification of genomic DNA,
reagents for the mass spectrometric analysis of nucleic acids.
